(19) United States
(12) Patent Application Publication
(10) Pub. No.: US 2011/0152510 A1
(43) Pub. Date: Jun. 23, 2011

(54) SIMPLE LOAD AND ELUTE PROCESS FOR PURIFICATION OF GENOMIC DNA

(75) Inventors: Sudhakar Rao Takkellapati, Walpole, MA (US); Manzer Khan, Union, NJ (US); Rajesh Ambat, Edison, NJ (US)

(73) Assignee: GE HEALTHCARE BIO-SCIENCES CORP., PISCATAWAY, NJ (US)

(21) Appl. No.: 13/059,992

(22) PCT Filed: Aug. 21, 2009
(86) PCT No.: PCT/US09/54557

§ 371 Date: Feb. 21, 2011

Related U.S. Application Data
(60) Provisional application No. 61/091,573, filed on Aug. 25, 2008.

(51) Int. Cl. C07H 21/04

(52) U.S. Cl. 536/23.1

(57) ABSTRACT

Provided is a novel two step chromatographic purification process (load and elute) for the isolation of genomic DNA. In this method the sample is loaded on the column and the genomic DNA product is eluted directly without any intermediate wash steps. This is accomplished by utilizing a restricted access resin (i.e., lid beads), which is easy to prepare and comprised of two layers with different properties with non-functional surfaces on the outer layer. The inner layer is modified with functional groups that act as ion-exchangers. Small molecules such as RNA and proteins can enter the inner part of the resin and larger genomic DNA molecules will pass through the resin. RNA and proteins are captured in the inner layer of the restricted access resin while genomic DNA is readily eluted in the flow-through.

### CROSS REFERENCE TO RELATED APPLICATIONS

This application is a filing under 35 U.S.C. §371 and claims priority to international patent application number PCT/US2009/054557 filed Aug. 21, 2009, published on Mar. 11, 2010 as WO 2010/027696, which claims priority to U.S. provisional patent application No. 61/091,573 filed Aug. 25, 2008; the disclosure of which is incorporated herein by reference in its entirety.

### FIELD OF THE INVENTION

This invention relates generally to methods for the separation and isolation of nucleic acids from a biological sample. In particular, the invention relates to a simple chromatographic process for the purification of genomic DNA from a mixture including other components.

### BACKGROUND OF THE INVENTION

In the last three decades there has been considerable effort in the development of improved methods for isolation and purification of nucleic acids and proteins from biological sources. This is mainly due to the increasing applications of nucleic acids and proteins in medicine and biological sciences. The genomic DNA isolated from blood, tissue or cultured cells have several applications, which include PCR, sequencing, genotyping, hybridization and southern blotting. Plasmid DNA has been utilized in sequencing, PCR, in the development of vaccines and in gene therapy. The isolated RNA has a variety of downstream applications, including blot hybridization, in vitro translation, cDNA synthesis, and RT-PCR. Similarly, the isolated protein can be used for Western Blot, DNA-protein interaction, enzymatic activity analysis, protein-protein interaction, and expression analysis.

Currently several commercial products are available for purification of DNA (genomic and plasmid), RNA and proteins. They utilize either silica based membrane purification, ion-exchange resins, charge switch technology or magnetic bead based purification. All these different technologies utilize the same chromatography principle of loading, washing off the impurities and eluting of the desired product(s). For example, for isolating genomic DNA from cells, the first step of the purification process is loading a crude cellular lysate on the column. In the second step the impurities are washed out from the bound DNA using an appropriate wash buffer. Finally DNA is eluted using an elution buffer. Despite the innumerable reports published in this area during the past 30 years, it still remains a complex and difficult task to separate negatively charged nucleic acids from each other and from other negatively charged components such as proteins.

For nucleic acid separation two main principles were previously used:
1. The separation medium has a firmly attached ligand structure to which desired nucleic acids and impurities have different abilities to become bound to or desorbed from. Typically the ligand structure is an anion exchange group and the separation is based on ion exchange, e.g. ion exchange chromatography (IEC) (For references, see those mentioned in US patent application US 2005/0267295).
2. The separation medium has a pore size permitting easier transport of one of desired nucleic acids or impurities within the pores. The separation is performed as a gel filtration (GF) (ibid).

Separation media which have an interior part and an outer surface layer with different separation functionalities were previously described. Some of these media carries a shielding layer (outer layer, lock, lid) which hinders passage of larger molecules into the interior part of the adsorbent matrix (sometimes called restricted access beads). It is suggested that these media can be used for the separation of proteins, plasmids, carbohydrates, lipids etc. WO 1998/039094, WO 1998/039364, US 2005/0267295, U.S. Pat. No. 7,208,093, and Gustavsson P.-E. et al. Journal of Chromatography A, 1038: 131-140 (2004). However, none of these discloses or teaches a simple process for the purification of genomic DNA.

There is a need for a simple and effective method for the purification of genomic DNA.

### SUMMARY OF THE INVENTION

It is an objective of the invention to provide methods for the purification of genomic DNA, which methods are improved with respect to (a) simplicity of operation, (b) increased purity and yield of genomic DNA and (c) a reduction of the number of steps involved.

The invention therefore provides a novel two step chromatographic purification process (load and elute) for the purification of genomic DNA. In this new purification method the sample is loaded on the column and the genomic DNA product is eluted directly without any intermediate wash steps. This simplified purification method is accomplished by utilizing a restricted access resin (i.e., lid beads), which is easy to prepare and comprised of two layers with different properties with non-functional surfaces on the outer layer. The inner layer is modified with functional groups that act as ion-exchangers. Small molecules such as RNA and proteins can enter the inner part of the resin and larger genomic DNA molecules will pass through the resin. RNA and proteins are captured in the inner layer of the restricted access resin while genomic DNA is readily eluted in the flow-through.

### BRIEF DESCRIPTION OF THE DRAWINGS

FIG. 1 shows an electrophoresis gel image of genomic DNA eluted in a proof of principle study described in Example 1.

FIG. 2 presents an electrophoresis gel image of genomic DNA isolated from blood sample, using the method described in Example 2.

### DETAILED DESCRIPTION OF THE INVENTION

A first aspect of the invention is a simple two step chromatographic purification process for genomic DNA from a sample containing other biomolecules which has a different size but affinity for the same ligand structure. In other words the method means separation of genomic DNA from other biomolecules which differ in size but have affinity for a common ligand structure.

The method thus comprises the steps of providing a sample solution containing genomic DNA and other biomolecule components; contacting the sample with a separation matrix to allow the other components to bind; and collecting the liquid phase which contains purified genomic DNA. If necessary, the genomic DNA recovered can be further purified.

The characteristic features of the inventive method are that the separation matrix used has (a) an interior part which carries a ligand structure which is capable of binding to both genomic DNA and other components, and is accessible to the other components; and (b) an outer surface layer that does not substantially adsorb genomic DNA, and is more easily penetrated by the other components than genomic DNA.

This means that the outer surface layer is accessible to substances in the sample by convective mass transport, and that the interior part of the matrix is only accessible via diffusive mass transport. The outer surface layer may thus be considered as a border-layer limiting a convective environment from a diffusive environment.

The outer surface layer may be located to the outer surface of porous particles or to the surface of macropores within particles or within monoliths comprising both macropores and micropores. The pores, at least in the outer surface layer, have a molecular size cut-off value for influx of compounds corresponding to an apparent molecular size between the apparent molecular sizes (hydrodynamic radius) for genomic DNA and other cellular components (e.g., RNA and proteins). The interior part may have pores with molecular cut-off values that are the same as pores in the outer surface layer, or have pores that are larger or smaller than these pores. The interior part may also contain a combination of these pore sizes.

The expression “an outer surface layer that does not substantially adsorb genomic DNA” means that at least the surface of the layer is essentially free from adsorptive ligand structures.

The outer surface layer may also contain repelling structures, e.g. structures of the same charge as genomic DNA; hydrophobic structures, etc. Repelling structures may improve the selectivity in transport through the outer surface layer. See WO 1998/039364.

The expression “is more easily penetrated by the other components than genomic DNA” means that the other components are transported substantially faster through the outer surface layer than genomic DNA. This includes that genomic DNA is completely excluded from the interior part.

The expression “carries a ligand structure which is capable of binding to both genomic DNA and other components” means that each of the substances is capable of binding to the ligand structure if they have had access to it. It follows that the difference in selectivity between genomic DNA and other components for binding to the bead is primarily caused by the pore size of the outer surface layer and not by a difference in the affinity as such for the ligand structure.

### The Sample

The sample can be derived from different sources and prepared in various ways. It may be derived from a blood sample, tissue sample, cultured cells etc. It may be in the form of a crude cell extract or a cell lysate. It may also be a processed sample that has undergone centrifugation, filtration, ultrafiltration, dialysis, precipitation etc for removing particulate matters, proteins, certain fractions of nucleic acids, concentration, desalting etc.

Thus, although optional, it is common practice to
(a) precipitate sample proteins before capturing and/or fractionating nucleic acids on an adsorbent,
(b) precipitate or degrade RNA, if a DNA fraction is to be isolated,
(c) reduce the ionic strength by desalting and/or diluting in case the sample is to be applied to an ion exchanger etc.

Other methodologies may also be applied in order to remove disturbing substances. In many cases the sample to be used in the instant invention is essentially free of particulate matters.

The sample typically is aqueous.

The other components separated from genomic DNA may be RNA or any other compound as long as it comprises a structure that is capable of binding to the ligand structure. This means that the other components may be a protein/polypeptide, or a carbohydrate, a lipid, a detergent, a cell or a part thereof etc. In important variants of the invention the other components comprise nucleic acid structure (oligonucleotide and RNA).

The apparent molecular size of a substance is determined by (a) its molecular weight, and (b) its shape under the condition applied. The apparent size may thus change upon change of pH, ionic strength, type of salt and temperature. This is in particular true for biopolymers such as high molecular weight nucleic acids and proteins. Matching of pore sizes within the interior part and within the outer surface layer with apparent sizes of desired molecules is easily done by testing the molecular size exclusion behavior of different interior parts and shielding layer (outer pores). It will also be possible to draw conclusions from the size exclusion behavior of the substances concerned on various size exclusion separation media. Common knowledge from size exclusion chromatography applies.

By properly setting the molecular size cut-off value of the outer surface layer, the contacting step of the present invention will facilitate separations of the sample into two fractions. One fraction contains genomic DNA of apparent sizes above the molecular size cut-off value. The second fraction contains other components having sizes below the molecular size cut-off value. One can thus envisage that the invention will render it possible to separate genomic DNA of varying length from smaller fragments of DNA, RNA etc. Typically the most useful molecular size cut-off values for the purification of genomic DNA will be in the interval corresponding to the apparent molecular sizes for useful genomic DNA, i.e. in the interval 1-20 kbp (kilo base pairs). This does not exclude that the cut-off value can be larger in case larger molecules are allowed to penetrate the interior part, for instance the interval may correspond to genomic DNA with a length of from 1 to 40 kbp.

In the preferred mode of the instant invention, the molecular size cut-off value of the outer surface layer is set so that the desired genomic DNA is retained in the liquid, i.e. not transported to any significant extent into the interior part of the matrix. One of the main advantages is that the genomic DNA then does not need to go through an adsorption/desorption process that may reduce yield and cause denaturation/degradation of the DNA.

### The Separation Matrix

The invention utilizes, as the separation matrix, porous polymeric particles which are comprised of two layers with different properties. In a specific embodiment, the particles present a neutral i.e. non-charged or non-functional outer layer. In certain preferred embodiments, the particles are produced according to the method described in US patent application publication US 2005/0267295, or alternatively, according to the method described in U.S. Pat. No. 7,208,093, the disclosures of which are hereby incorporated-by-reference in their entirety.

The separation matrix has an interior part (i.e., inner pores) carrying a ligand structure which is capable of binding to both genomic DNA and other components, and is accessible to the other components; and an outer surface layer that does not substantially adsorb genomic DNA, and is more easily penetrated by the other components than genomic DNA. This means that the outer surface layer is accessible to substances in the sample by convective mass transport, and that the interior part of the matrix is only accessible via diffusive mass transport. The outer surface layer may thus be considered as a border-layer limiting a convective environment from a diffusive environment.

### (1) The Ligand Structure

The ligands that are coupled to the surfaces of the inner pore system can be any well-known groups conventionally used as ligands in chromatography, or a combination thereof, such as affinity groups, hydrophobic interaction groups, ion-exchange groups, such as negatively charged cation-exchange groups or positively charged anion exchange groups, etc. Thus, in the present context, the term “binding” refers to any kind of adsorption or coupling. Accordingly, in one embodiment of the present method, the binding groups are ion-exchange groups. In a specific embodiment, the anion exchanger is diethylamine (ANX) or ethylenediamine (EDA).

The most apparent ligand structures for the current applications contain positively charged groups (anion exchanging groups). Anion exchanging groups in principle bind to any negatively charged species. Therefore, these kinds of ligand structures may be used in the instant invention for separating any negatively charged species from genomic DNA. The only demand is that the difference in apparent molecular size shall be sufficiently large. One and the same matrix may contain two or more different ligands, for instance anion exchange ligands.

The preferred anion exchange ligands provide mixed mode interaction with the substance to be bound and/or allow for decharging by a pH-switch (increase in pH) at moderate alkaline pH-values. The ability of decharging means that the anion exchange ligands comprise primary, secondary and tertiary ammonium groups, with preference for those having pKa ≦10.5 or ≦10.0, i.e. typical primary or secondary ammonium groups. In the variants believed to be most preferred, essentially all anion exchange groups should comply with this criterion.

The term “the anion exchange ligand provides mixed mode interaction with the substance to be bound” refers to a ligand that is capable of providing at least two different, but co-operative, sites which interact with the substance to be bound. One of these sites gives an attractive type of charge-charge interaction between the ligand and the substance of interest. The second site typically gives electron donor-acceptor interaction including hydrogen-bonding.

Electron donor-acceptor interactions mean that an electronegative atom with a free pair of electrons acts as a donor and bind to an electron-deficient atom that acts as an acceptor for the electron pair of the donor. See Karger et al., An Introduction into Separation Science, John Wiley & Sons (1973) page 42.

### (2) The Interior Part of the Matrix

This part of the matrix is typically of the same type as commonly utilized within affinity adsorption such as chromatography. The interior part may comprise both macropores and micropores.

The interior part is preferably hydrophilic and in the form of a polymer, which is insoluble and more or less swellable in water. Hydrophilic polymers typically carry polar groups such as hydroxy, amino, carboxy, ester, ether of lower alkyls (such as (—CH₂CH₂O—)ₙH, (—CH₂CH(CH₃)O—)ₙH, and groups that are copolymerisates of ethylene oxide and propylene oxide (e.g. PLURONICS™) (n is an integer >0, for instance 1, 2, 3 up to 100). Hydrophobic polymers that have been derivatized to become hydrophilic are also included in this definition. Suitable polymers are polyhydroxy polymers, e.g. based on polysaccharides, such as agarose, dextran, cellulose, starch, pullulan, etc. and completely synthetic polymers, such as polyacrylic amide, polymethacrylic amide, poly(hydroxyalkyl vinyl ethers), poly(hydroxyalkylacrylates) and polymethacrylates (e.g. polyglycidylmethacrylate), polyvinylalcohols and polymers based on styrenes and divinylbenzenes, and copolymers in which two or more of the monomers corresponding to the above-mentioned polymers are included. Polymers, which are soluble in water, may be derivatized to become insoluble, e.g. by cross-linking and by coupling to an insoluble matrix via adsorption or covalent binding. Hydrophilic groups can be introduced on hydrophobic polymers (e.g. on copolymers of monovinyl and divinylbenzenes) by polymerization of monomers exhibiting groups which can be converted to OH, or by hydrophilization of the final polymer, e.g. by adsorption of suitable compounds, such as hydrophilic polymers.

The interior part can also be based on inorganic material, such as silica, zirconium oxide, graphite, tantalum oxide etc. The interior part is preferably devoid of hydrolytically unstable groups, such as silan, ester, amide groups and groups present in silica as such. In a particularly interesting embodiment of the present invention, the interior part is in the form of irregular or spherical beads with sizes in the range of 1-1000 μm, preferably 5-1000 μm. The interior part may also be in the form of a porous monolith.

The ligand structures are introduced into the interior part by methods known in the field. The required degree of substitution for ligand structures (density of ligand structures) will depend on ligand type, kind of matrix, compound to be bound etc. Usually it is selected in the interval of 0.001-4 mmol/ml matrix, such as 0.01-1 mmol. For agarose-based matrices the density is usually within the range of 0.1-0.3 mmol/ml matrix. For dextran based matrices the interval may be extended upwards to 0.5-0.6 mmol/ml matrix.

The ranges given in the preceding paragraph refer to the capacity for the matrix in fully protonated form to bind chloride ions. “ml matrix” refers to the matrix saturated with water. The outer surface layer is included in the matrix in calculating these ranges.

### (3) The Outer Surface Layer

The outer surface layer (shied, lock) must be penetrable by the liquid sample. For aqueous liquid this means that the outer surface layer should be built up of a hydrophilic polymer.

There are different methodologies for creating the outer surface layer. One of the methods includes coating the surface of a naked form of a porous particle or the surfaces of macropores of particles or of a monolith which have both macropores and micropores with a hydrophilic polymer. The apparent molecular size of the hydrophilic polymer should be selected such that it cannot significantly penetrate the pores that are aimed at being part of the interior. Preferably the hydrophilic polymer comprises hydrophilic groups as discussed above, e.g. is a polyhydroxy polymer such as polysaccharides in soluble forms (dextran, agarose, starch, cellulose etc). The ligand structures may be introduced onto the interior part either before or after creation of the outer surface layer. The permeability for various substances of the outer surface layer produced in this way will be controlled by the concentration and size of the polymer in the solution used for coating. Subsequent to coating the outer surface layer may be stabilized by cross-linking within the layer as well as to the interior part. This methodology is described in detail in WO 1998/039094 (Amersham Pharmacia Biotech AB).

The lock medium used in the present invention may be in the form of particles/beads that have densities higher or lower than the liquid (for instance by introducing one or more density-controlling particles per matrix particle). This kind of matrix is especially applicable in large-scale operations for fluidized or expanded bed chromatography as well as different batch-wise chromatography techniques in non-packed columns, e.g. simple batch adsorption in stirred tanks. These kinds of techniques are described in WO 1992/018237 (Amersham Pharmacia Biotech AB) and WO 1992/000799 (Kem-En-Tek/Upfront Chromatography) and can easily be adapted to the inventive concept by introducing a lock on the particles used.

### Other Considerations

The conditions for running the inventive process are in principle the same as for conventional adsorption techniques, e.g. anion exchange chromatography. Because of the unique structure of the separation matrix, smaller nucleic acid molecules (e.g., RNA) and proteins enter the interior part of the matrix while genomic DNA does not. The genomic DNA is readily collected in the liquid phase. In a preferred method, the separation matrix is in the form of a packed bed column and the genomic DNA is collected right off the column, in the flow-through. This method offers the simplest process for separation and isolation of genomic DNA from cellular contaminants. The process only takes between 5-10 minutes as compared to other available methods which take at least 30-40 minutes to complete.

In addition to the separation and purification of genomic DNA from cellular contaminants, this process can also be used to remove impurities present in genomic DNA purified by other methods, including RNA and other impurities. This is sometimes referred as a polishing step. Here, desalting is also achieved simultaneously for the genomic DNA.

Since the purification process presented here is the simplest genomic DNA purification process this can also be explored in the development of automation.

By carefully choosing the ligand structure, certain components that associate with the interior part of the matrix can be eluted. Desorption from the matrix is accomplished by increasing the ionic strength of the liquid in contact with the matrix until the desired component(s) is eluted. In particular in case the ligand structure is the protonated form of a primary, secondary or tertiary amine group and/or desired component is a nucleic acid (e.g., RNA), desorption is preferably assisted by increasing the pH. An alternative method for desorption is to include a soluble ligand analogue in the liquid, i.e. a structure analogue that is able to compete with the ligand structure for binding to the desired component. The presence of structure-breaking compounds in the liquid may also assist desorption. This in particular may apply in case the ligand structure contains one or more hydroxyl group or amino group at a carbon atom at 2 or 3 atoms distance from a charged primary, secondary or tertiary nitrogen of the ligand structure. Well-known structure breaking agents are guanidine and urea. See also WO 1997/029825 (Amersham Pharmacia Biotech AB). Therefore, also provided is a simple process for the isolation of both genomic DNA and other components such as RNA or proteins from one sample.

As indicated above the isolated genomic DNA and other components may be further purified, for instance by so called polishing and or intermediate purification steps. The need for extra purification/polishing steps typically applies if the purity demand on the desired substance is high, such as for in vivo therapeutics. Such additional steps may involve adsorbtion/desorption to/from an anion exchanger, a cation exchanger, a reverse phase matrix, a HIC matrix (hydrophobic interaction chromatography matrix) etc. Size exclusion chromatography and adsorption/desorption on hydroxy apatite may also be used.

### EXPERIMENTAL PART

Below, the present invention will be described by way of examples. However, the present examples are provided for illustrative purposes only and should not be construed as limiting the invention as defined by the appended claims. All references given below and elsewhere in the present specification are hereby included by reference.

### Example 1

### Evaluation of Lid Bead Resins for Genomic DNA Purification

In an effort to find a simple and effective method for the separation and purification of genomic DNA, we tested a variety of matrices. Two of them are the so called lid beads. One type of the lid beads was coupled with diethylamine (ANX), while the other type was coupled with octylamine (Octyl). The beads were made according to the methods disclosed in the Examples section of U.S. Pat. No. 7,208,093, the disclosure of which is hereby incorporated by reference in its entirety.

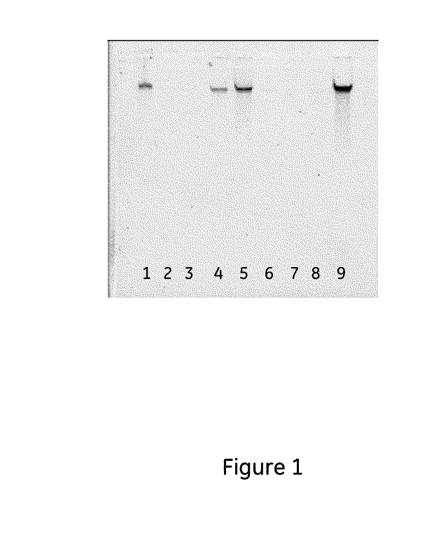

The beads were packed in a NAP™-10 column (GE Healthcare, Piscataway, N.J.) to a bed height of 1.2 cm with 1×TE buffer. 20 μg of genomic DNA in 1 ml of 1×TE buffer was loaded on each of the columns Load fraction and 2 ml of 1×TE elution were collected as the first fraction. Second elution was done with 3 ml of 1×TE and third elution with 2 ml of 1×TE. Final (fourth) elution was done with 2 ml of a solution containing 1M NaCl and 0.5M K₂CO₃. FIG. 1 shows an electrophoresis gel image of the genomic DNA collected in fractions one through four, using a column packed with the ANX matrix (lanes 1-4), or the Octyl matrix (lanes 5-8), compared to input genomic DNA as a control (lane 9).

Genomic DNA did not bind to the Octyl matrix (genomic DNA is present only in the first fraction, see lane 5). In case of ANX matrix, a portion of the input DNA was eluted in the beginning (lane 1), and the remaining DNA was eluted with a solution containing 1M NaCl and 0.5M K₂CO₃ (lane 4). To determine whether genomic DNA was bound to the matrix by non-specific interactions or by ion-exchange mechanism, experiments were performed with increasing amounts of salt concentration (100-500 mM). If the genomic DNA can be elution without very high salt concentration buffer, than the binding is only by non-specific interactions. Indeed we were able to elute most of the genomic DNA by using less than 200 mM salt buffer. Therefore genomic DNA interacts with the matrix in a non-specific manner and does not interact with the ligands in the interior part of the matrix.

We also carried out similar experiments using a mixture of RNA and genomic DNA. In these experiments, DNA was readily separated in the flow-through with no RNA contamination.

### Example 2

### Simple, Two Step Purification of Genomic DNA from Cell Lysate

Experiments were carried out to evaluate the lid bead matrix for purification of genomic DNA from cell lysate.

Human blood sample was lysed using a lysis protocol from the ILLUSTRA™ blood genomicPrep Midi Flow Kit (GE Healthcare, Piscataway, N.J.). Five milliliters of blood was processed by first isolating the white blood cells, then lysis of isolated white blood cells and incubation with Proteinase K (See pages 16-17 of the product booklet, Rev E 08/2007).

Columns were packed as in Example 1 above, using an Octyl-coupled lid resin pre-equilibrated in 1×TE buffer. One fifth of the lysate was diluted to one milliliter and loaded on to a column. The void volume was collected in an Eppendorf tube. Then 2.5 ml of 1×TE buffer was loaded onto the column and the flow-through was collected in 0.5 ml fractions.

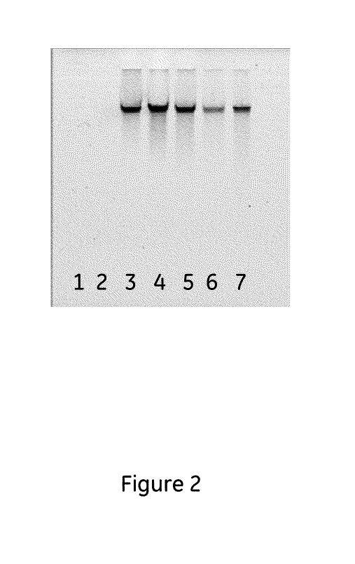

FIG. 2 presents an electrophoresis gel image of the collections. Lane 1 was from the void volume, while lanes 2-6 were from the flow-through collected, from the first fraction to the fifth fraction, in that order. Lane 7 was control genomic DNA isolated using the ILLUSTRA™ blood genomicPrep Midi Flow Kit. The results show that the void volume and the first fraction did not contain any genomic DNA. Fractions 2 to 4 (i.e., lanes 3-5) contain most of the product and without any RNA impurities. The purity of the material is comparable to the genomic DNA isolated using ILLUSTRA™ blood genomicPrep Midi Flow Kit (lane 7).

Fractions 2 and 3 (gel lanes 3, 4) contained the pure product without any salt (See Table 1: UV spectral data). The purity (UV 260/280) was in the specification range of 1.76-1.90, thus demonstrating that there was little protein contamination in the purified genomic DNA. Therefore, genomic DNA was separated from crude cell lysate and successfully isolated in a simple load/elute process.

TABLE 1
UV spectral data, 1xTE as reference

| | 230 | 260 | 280 |
|---|---|---|---|
| Fraction 2 | 0.1275 | 0.2625 | 0.1495 |
| Fraction 3 | 0.2547 | 0.4882 | 0.2729 |
| Fraction 4 | 1.6520 | 0.2087 | 0.1096 |

All patents, patent publications, and other published references mentioned herein are hereby incorporated by reference in their entireties as if each had been individually and specifically incorporated by reference herein. While preferred illustrative embodiments of the present invention are described, one skilled in the art will appreciate that the present invention can be practiced by other than the described embodiments, which are presented for purposes of illustration only and not by way of limitation. The present invention is limited only by the claims that follow.

We claim:

1. A method for purifying genomic DNA from a sample solution containing genomic DNA and other components, which method comprising:
(i) providing said sample solution containing genomic DNA and other components;
(ii) contacting said sample with a separation matrix to allow said other components to bind; and
(iii) collecting the liquid phase which contains purified genomic DNA;
wherein said separation matrix is a material having:
(a) an outer surface layer that does not substantially adsorb genomic DNA, and is more easily penetrated by the other components, and
(b) an interior part which
carries a ligand structure that is capable of binding to both genomic DNA and other components, and
is accessible to the other components.

2. The method of claim 1, wherein the sample solution is a clarified alkaline lysate.

3. The method of claim 1, wherein the sample solution is a mixture of biomolecules including genomic DNA.

4. The method of claim 1, wherein the outer surface layer is penetrable by other components but not by genomic DNA.

5. The method of claim 1, wherein the ligand structure includes a positively charged group.

6. The method of claim 5, wherein the positively charged group is selected from the group consisting of primary, secondary and tertiary ammonium groups.

7. The method of claim 5, wherein the positively charged group is a mixed mode anion exchanger.

8. The method of claim 1, wherein the outer surface layer is essentially free of ligand structures.

9. The method of claim 1, wherein the separation matrix is in the form of a packed chromatography column and the liquid phase collected is flow-through from the column.
